(19) United States
(12) Patent Application Publication
(10) Pub. No.: US 2012/0190703 A1
(43) Pub. Date: Jul. 26, 2012

(54) SALTS OF 13A-(S)DESOXYTYLOPHORININE, PREPARATION METHODS AND PHARMACEUTICAL COMPOSITIONS AND USES THEREOF

(75) Inventors: Shishan Yu, Beijing (CN); Xiaoguang Chen, Beijing (CN); Haining Lv, Beijing (CN); Yan Li, Beijing (CN); Song Xu, Beijing (CN); Shuanggang Ma, Beijing (CN); Zhenjia Liu, Beijing (CN); Yi Zhang, Beijing (CN); Jinping Hu, Beijing (CN)

(73) Assignee: INSTITUTE OF MATARIA MEDICA, CHINESE ACADEMY OF MEDICAL SCIENCES, Beijing (CN)

(21) Appl. No.: 13/382,701

(22) PCT Filed: Jul. 9, 2010
(86) PCT No.: PCT/CN2010/075083

§ 371 Date: Mar. 30, 2012

(30) Foreign Application Priority Data
Jul. 9, 2009 (CN) 200910089238.5

(51) Int. Cl. A61K 31/4745; A61P 35/00; A61P 29/00; C07D 471/04

(52) U.S. Cl. 514/283; 546/42

(57) ABSTRACT

The present invention relates to the salts of (+)-13a-(S)-deoxytylophorinine represented by the general formula (I), the preparation method thereof, the pharmaceutical compositions containing them, and their use as medicaments for prevention and/or treatment of cancer and/or inflammation disease.

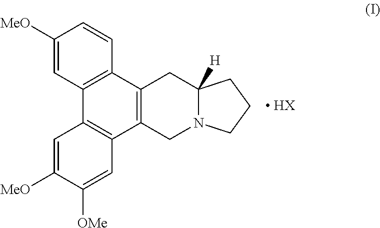

### TECHNICAL FIELD

The present invention relates to the salts of 13a-(S)-deoxytylophorinine, the preparation method thereof, the pharmaceutical compositions containing them, and their use as medicaments for prevention and/or treatment cancer and/or inflammation disease.

### BACKGROUND ART

Phenanthroindolizidine alkaloids are pentacyclic natural products isolated mainly in plants belonging to Tylophora as well as some genera of the Asclepiadaceae. These natural products attract widespread attention for their extensively therapeutic activities, especially anti-tumor activity and anti-inflammatory activity. Some of these alkaloids have been screened against 60 tumor cell lines by NCI of USA. It's found that some of these compounds display highly cytotoxic activity with GI₅₀ less than 10⁻⁸M. Furthermore, this activity exhibit highly selectivity to some malignant tumor cells, such as melanoma and lung cancer. These alkaloids are effective to drug-fast cancer cells without cross resistance with other anti-cancer drug.

(+)-13a-(S)-Deoxytylophorinine (Chinese patent application No. 200610076298.X, CN 101058578A), one of the phenanthroindolizidine alkaloids, was isolated from Tylophora atrofoculata and Tylophora ovata, which exhibit notable anti-tumor activity that IC₅₀ to Ketr3, HCT-8, A549, BGC-803, Bel-7402, B16BL6, KB, CaSE-17, HL-60 cells is 0.1 μM and 0.3 μM respectively. (+)-13a-(S)-Deoxytylophorinine displayed antitumor activity in vitro. It exhibited notable anti-tumor proliferative activity of mice H22 liver cancer and mice Lewis lung cancer and displayed good antitumor activity in vivo. The compound is not toxic in the liver and kidney in preliminary toxic experiments.

The inhibitory mechanisms studies illustrated that phenanthroindolizidine alkaloids could inhibit RNA, DNA and protein synthesis (Bioorg Med Chem. Lett. 2006, 16: 4300-4304.). But further studies indicated that the action mechanism of these compounds is completely different with clinically used anti-tumor drugs. These compounds can intensively inhibit NF-κB signal pathway that participate in RNA transcription procedure selectively (Mol Cancer Ther. 2006, 5(10):2484-2493.). But until now the specific target is still unknown.

The anti-inflammation activity of these alkaloids is also related with their inhibition to NF-κB signal pathway, which intensively correlative with the action that blocks MEKK1 action (Mol. Pharm. 2006, 69(3):749-758). But the specific target is unknown. 13a-(S)-Deoxytylophorinine is fat-insoluble higher, insoluble in water.

### SUMMARY OF THE INVENTION

The technological problem solved by the present invention is to provide salts of 13a-(S)-Deoxytylophorinine compound such as general formula (I), its pharmaceutically acceptable hydrate and prodrug.

Another technological problem solved by the present invention is to provide a preparation method of salts of 13a-(S)-Deoxytylophorinine compound such as general formula (I), its pharmaceutically acceptable hydrate and prodrug.

Another technological problem solved by the present invention is to provide pharmaceutical compositions comprising at least one of salts of 13a-(S)-Deoxytylophorinine compound such as general formula (I), its pharmaceutically acceptable hydrate and prodrug with pharmaceutical carrier and/or excipient.

Another technological problem solved by the present invention is to provide use of salts of 13a-(S)-Deoxytylophorinine compound such as general formula (I), its pharmaceutically acceptable hydrate and prodrug for the preparation of the medicaments for prevention and/or treatment cancer and/or inflammation diseases.

To solve the said problems above, the technological program the present invention applied is:

According to the present invention, salts of (+)-13a-(S)-Deoxytylophorinine represent as general formula (I):

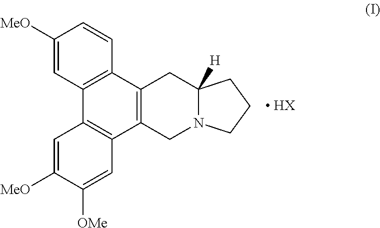

Wherein:

HX are organic acid or inorganic acid;

When HX are inorganic acid, them include, but are not limited to hydrochloric acid, sulfuric acid, phosphoric acid, hydrobromic acid, nitric acid;

When HX are organic acid, them include, but are not limited to tartaric acid, citric acid, maleic acid, lactic acid, salicylic acid, malic acid, benzoic acid, hexanedioic acid, fumaric acid, succinic acid, sulfonic acid, alginic acid, amino acid, acetyl benzoic acid, folic acid, N-cyclohexylamino sulfonic acid, polygalacturonic acid, methane sulfonic acid, tosic acid, citric acid.

Preferable organic acid are selected from tartaric acid, citric acid, maleic acid, lactic acid, salicylic acid, malic acid, benzoic acid, hexanedioic acid, fumaric acid, succinic acid.

In order to achieve the object of the present invention, preferable salts of (+)-13a-(S)-Deoxytylophorinine include, but are not limited to the compounds represent as general formula (IA):

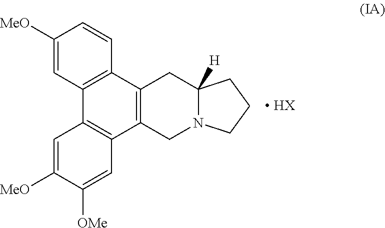

Wherein:

preferable HX are selected from tartaric acid, citric acid, maleic acid, lactic acid.

In order to achieve the object of the present invention, preferable salts of (+)-13a-(S)-Deoxytylophorinine include, but are not limited to the following compounds:

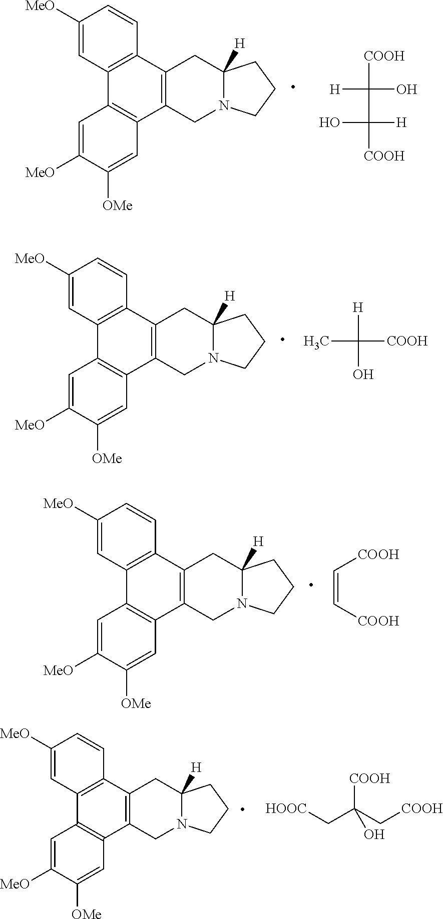

The present invention provides the preparation method of invention compounds:

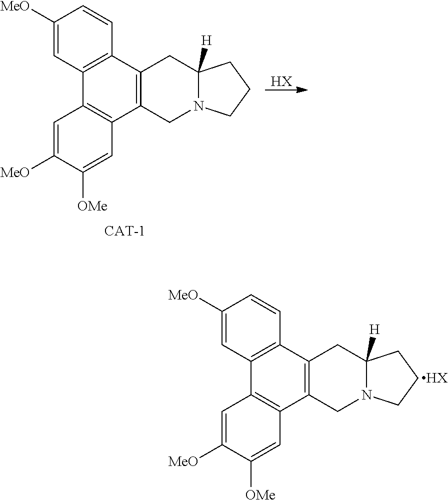

Details of preparation method include several procedures below:

Equivalent (+)-13a-(S)-Deoxytylophorinine (CAT-1) and corresponding acid are suspended in anhydrous ethanol. The mixture is stirred at 50° C. for 30 min until become clear. The reactant solvent is cold to room temperature and evaporated to get solid salt.

The salt prepared can be purified by recrystallization from ethanol.

(+)-13a-(S)-Deoxytylophorinine can be prepared according to the method of

[symbol] 13a-(S)-deoxytylophorinine, preparation methods, compositions, and uses [symbol] (Chinese patent application No. 200610076298.X) and [symbol] 13a-(S)-deoxytylophorinine analogue, preparation methods, compositions, and uses [symbol] (Chinese patent application No. 200910079163.2).

The present invention provides a pharmaceutical formulation comprising therapeutically effective amount of (+)-13a-(S)-Deoxytylophorinine salt of general formula (I) and pharmaceutical acceptable carrier.

The present invention relates to pharmaceutical formulation containing a compound of invention as active component, and pharmaceutical excipient or adjuvant. The normal content of the compound of invention in the pharmaceutical formulation of invention is 0.1˜95% (g/g). The normal content of the compound of invention in a unit dosage form is 0.1˜100 mg, the optimized content is 4˜50 mg.

The pharmaceutical formulation may be prepared according to customary methods. For this reason, if required, the compound of invention could be prepared as human or veterinary medicine combined with one or more solid or liquid pharmaceutically acceptable excipient and/or adjuvant as administering form or dosage form.

The compound of invention or its pharmaceutical formulation could be administered as unit dosage form. The administering route could be intestinal tract or others, such as muscle, subcutaneous tissue, nasal cavity, oral mucosa, skin, abdominal membrane, intestinum rectum.

The compound of invention or its pharmaceutical formulation could be administered by injection, including intravenous injection, intramuscular injection, hypodermic injection, endodermic injection, injectio ad acumen.

The administration form could be presented as form of liquid and solid. The liquid form could be solution, colloid, particulate, emulsion, suspension. Others could be present as the form of tablets, capsules, drop pills, aerosols, pills, powder, solution, suspension, emulsion, granules, suppositories, freeze-dried injectable powder, etc.

The compound of present invention could be prepared as general preparation, and also could be prepared as sustained release preparation, controlled release preparation, targeting preparation, and all kinds of particulate delivery system.

General customary pharmaceutical carrier could be used in the preparation of tablets from unit administering dosage form of the compound of invention. The particular example of the carrier, such as diluent agent and absorption agent is starch, dextrin, calcium sulfate, lactose, mannitol, sucrose, sodium chloride, glycose, urea, Calcium Carbonate, porcellanite, microcrystalline cellulose, aluminium silicate, etc. The particular examples of wetting agent and adhesive is water, glycerol, polyglycol, ethanol, propanol, starch paste, dextrin, syrup, honey, glycose slution, acacia mucilage, gelatin mucilage, CMC-Na, shellac, methyl cellulose, tribasic potassium phosphate, plasdone, etc. The particular example of disintegrating agent is dry starch, alginate, powdered agar, laminarin, bicarbonate of soda and citric acid, calcium carbonate, fatty acid ester of polyoxyethylene sorbitan, sodium dodecylsulphate(SDS), methyl cellulose, ethyl cellulose, etc. The particular example of disintegrating depressor agent is sucrose, tristearin, cacao butter, hydrogenated oil, etc. The particular example of absorption enhancer is quaternary ammonium salt, sodium dodecylsulfate, etc. The particular example of lubricant is chalk, silicon dioxide, corn starch, stearate, boracic acid, liquid paraffin, polyglycol, etc. The tablet could be further prepared as coated tablet, such as sugar coated tablet, film coated tablet, enteric-coated tablet, double layer tablet and poly layer tablet.

General customary pharmaceutical carrier could be used in the preparation of pills from unit administering dosage form of the compound of invention. The particular example of the carrier, such as diluent agent and absorbent is glycose, lactose, starch, cacao butter, hydrogenated vegetable oil, PVP, glycerolipid ester of monostearate, kaolin, chalk, etc. The particular example of bonding agent is acacia, gum dragon, gelatin, ethanol, honey, liquid sugar, panada, etc. The particular example of disintegrating agent is powdered agar, dry starch, alginate, sodium dodecylsulphate(SDS), CMC-Na, methyl cellulose, methyl cellulose, ethyl cellulose, etc.

To prepare capsules as unit dosage form, the mixture of active compound of present invention and pharmaceutical carrier said above would be filled in hard gelatin capsule or soft capsule after well-distributed blending. The active compound could also fiat microcapsule and suspended in aqueous medium as suspension, or filled in hard capsule, or as injection.

The compound of present invention could be prepared as injection, such as solution, suspension, emulsion, freeze-dried injection powder. The preparation could be aqueous or nonaqueous that comprising one and/or more pharmaceutically acceptable carrier, diluent agent, bonding agent, lubricant, conservative, surfactant or dispersing agent. The particular example of diluent agent is water, ethanol, polyglycol, trimethylene glycol, ethoxyl-isooctadecanol, polyoxide of isooctadecanol, fatty acid ester of polyoxyethylene sorbitan, etc. Sodium chloride, glucose and glycerol could be added to the injection to make it isotonic. The preparation could also contain one or more agents such as auxiliary solvent, buffer, pH regulator, which is customary used in this field.

Some other agents could be added to the preparation such as colorings, conservatives, flavoring, correctant, sweeteners or others if required.

To achieve and reinforce the therapeutic efficacy, the compound and formulation of present invention could be administered by any known route.

The dose of the compound and formulation of present invention depend on many factors, such as the kind and severity of the disease, sexuality, age, weight, character and individual response of the patients or animals, administering route, administering frequency and goals of treatment. Therefore the administering dose of the compound and formulation could be changed in a large scope. Generally say, the dosage of active component of present invention is generally accepted by the technicians of this field. In order to meet the requirement of efficacy and achieve the prophylactic or therapeutic purpose of present invention, the administering dose could be adjusted according to the actual content of the compound in the formulation of present invention. The suitable dosage scope of the compound of the invention is 0.001˜100 mg/Kg body weight per day, the optimized dose is 0.1˜60 mg/Kg body weight, the better choice is 1˜30 mg/Kg body weight, the best choice is 2˜15 mg/Kg body weight. The suitable dosage scope of the compound of the invention for adult patient is 10˜500 mg per day, the optimized dose is 20˜100 mg, and could be administered in one time or divided into 2˜3 times. The suitable dosage scope for child patient is 5˜30 mg/Kg body weight, the optimized dose is 10˜20 mg/kg body weight. The compound of the said dose above could be administered by single dose pattern or divided into two, three or four dose pattern, which is limited by the clinic experiences and administering program of the doctor. The compound of present invention could be administered alone, or combined with other drugs.

The present invention related to the application of the compound of invention in preventing and/or treating cancer and/or inflammation disease. The particular examples of said cancer comprising human colon cancer, human stomach cancer, human ovarian cancer, human uterine cervix cancer, human liver cancer, human lung cancer, human pancreatic cancer.

The technological forte of present invention is:

(+)-13a-(S)-Deoxytylophorinine can not be dissolved in water due to its high liposolubility. The water-solubility is elevated after salification.

The anti-tumor activity of (+)-13a-(S)-Deoxytylophorinine salt is equivalent to (+)-13a-(S)-Deoxytylophorinine according to the result of in vitro activity assay and pharmacodynamic experiment, which means the anti-tumor activity is retained after salification.

As the results of plasma pharmacokinetics study with the salts of tartaric acid, citric acid and maleic acid revealed, the pharmacokinetics qualities of (+)-13a-(S)-Deoxytylophorinine was optimized with faster absorption, shorter peak time. Moreover, the MRT of the salts in vivo was obviously prolonged than (+)-13a-(S)-Deoxytylophorinine. The AUC of the salts was equivalent or elevated with (+)-13a-(S)-Deoxytylophorinine such as maleic acid salt. After oral administration of tartrate, citrate and maleate, the contents of tartrate and maleate in experimental animal brain tissue were higher than their prototype at three time points.

### ILLUSTRATION OF THE FIGURES

FIG. 1 Impact of (+)-13a-(S)-deoxytylophorinine and its salts on the Growth of Cancer Xenografts (H22) in Mice.

FIG. 2 Impact of (+)-13a-(S)-Deoxytylophorinine and its salt on the body weight of Kcnming (KM) mice.

FIG. 3 Impact of (+)-13a-(S)-Deoxytylophorinine and its salt on the Growth of Cancer Xenografts (H22) in Mice.

FIG. 4 Standard curve of (+)-13a-(S)-deoxytylophorinine in mice plasma

FIG. 5 The concentration-time curve of (+)-13a-(S)-Deoxytylophorinine (6 mg/kg) and its three salts in plasma after oral administration.

FIG. 6 Contents of (+)-13a-(S)-deoxytylophorinine tartrate in brain tissue after oral administration.

FIG. 7 Contents of (+)-13a-(S)-deoxytylophorinine maleate in brain tissue after oral administration.

FIG. 8 Contents of (+)-13a-(S)-deoxytylophorinine citrate in brain tissue after oral administration.

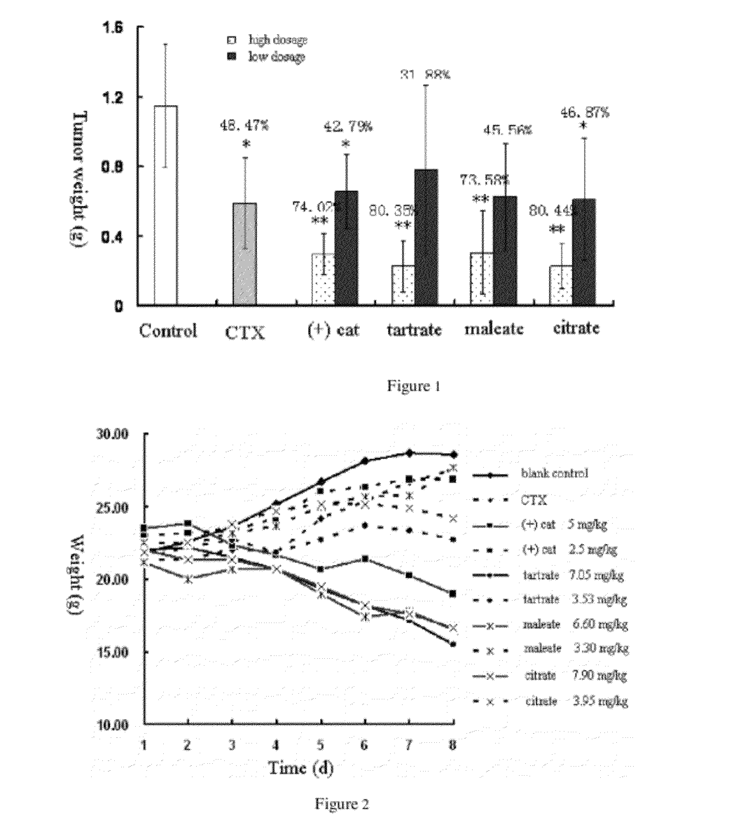

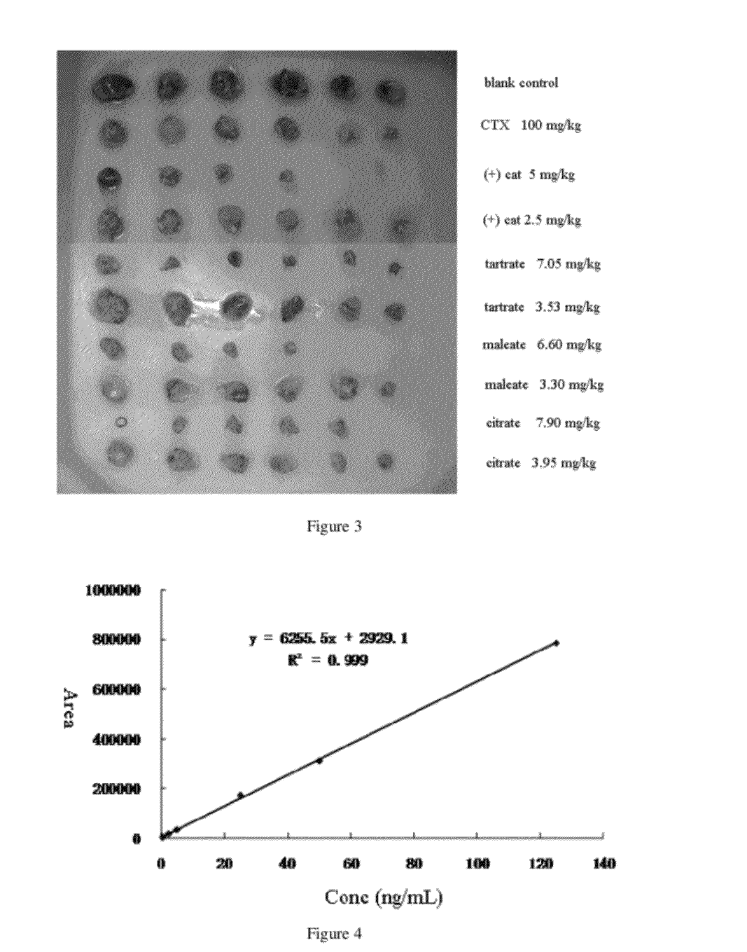

### EXAMPLES

The used starting chemical compounds of the detailed description of present invention could be prepared by customary methods that well known for the technicians of this field, and could be prepared by the method of descriptions below. The purpose of the detailed description below is to illustrate the present invention further, but not means any limitations to this invention.

### ABBREVIATION

### CTX: Cytoxan

### DMSO: Dimethyl Sulfoxide

MRT(0-∞): Mean residence time
AUC(0-∞): Biological availability
t1/2z: Half-life period

### Tmax: Peak Time

Cmax: Peak concentration
+CAT: (+)-13a-(S)-Deoxytylophorinine

### Synthesis Example

### The synthesis of (+)-13a-(S)-deoxytylophorinine

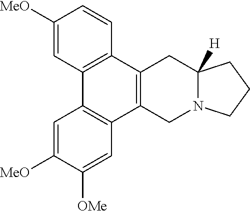

(+)-13a-(S)-deoxytylophorinine was synthesized according to the method of patent [symbol] 13a-(S)-deoxytylophorinine, its preparation, pharmaceutical formulations and application [symbol] (application number 200610076298.X) and [symbol] 13a-(S)-deoxytylophorinine analogue, its preparation, pharmaceutical formulations and application [symbol] (application number 200910079163.2). [α]_{D}²⁰ (c=0.25, CHCl₃)=+102°, ee=99.1% [AD-H; isopropanol:hexane (15:85), 0.1% TEA; λ=254 nm; t(major)=18.79 min, t(minor)=26.09 min], ESI-MS: 364.2 [H+H]⁺, ¹H-NMR (400 MHz, CDCl₃): 7.92 (1H, d, J=9.2 Hz, H-1), 7.21 (1H, dd, J=9.2 Hz, 2 Hz, H-2), 7.88 (1H, d, J=2 Hz, H-4), 7.90 (1H, s, H-5), 7.12 (1H, s, H-8), 4.00 (3H, s, MeO), 4.04 (3H, s, MeO), 4.09 (3H, s, MeO), 4.62 (1H, d, J=14.8 Hz, H-9), 3.71 (1H, d, J=14.8 Hz, H-9), 3.45 (1H, m, H-10), 3.39 (1H, m, H-10), 2.98 (1H, m, H-14), 2.57 (1H, m, H-13a), 2.53 (1H, m, H-10), 2.24 (1H, m, H-12), 2.04 (1H, m, H-11), 1.93 (1H, m, H-11), 1.78 (1H, m, H-12), ¹³C-NMR (100 MHz, CDCl₃): 125.11 (C-1), 114.83 (C-2), 157.61 (C-3), 104.55 (C-4), 103.92 (C-5), 149.42 (C-6), 148.30 (C-7), 103.02 (C-8), 53.46 (C-9), 54.91 (C-10), 21.53 (C-11), 31.03 (C-12), 60.15 (C13a), 33.03 (C-14), 55.48 (C3-OMe), 55.96 (C6-OMe), 55.90 (C7-OMe), 123.34, 125.41, 125.42, 126.78, 130.39. HRMS (ESI) calcd for [M]⁺ C₂₃H₂₆NO₃ 363.1834, found 363.1852.

### Example 1

### Preparation of (+)-13a-(S)-deoxytylophorinine Tartrate

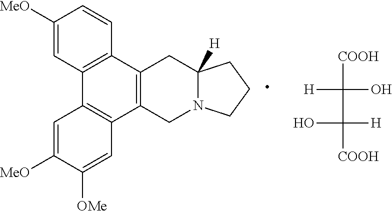

20 mg of (+)-13a-(S)-Deoxytylophorinine (CAT-1) and 8.26 mg of tartric acid is suspended in 5 mL of ethanol. The mixture is stirred for 10 min in reflux. The reactant solvent is cold to room temperature and evaporated to 2 mL with white solid precipitated. After filtration 22 mg of the tartrate salt was obtained. Mp: 216˜218° C. The product is soluble in water.

### Example 2

### Preparation of (+)-13a-(S)-deoxytylophorinine Maleate

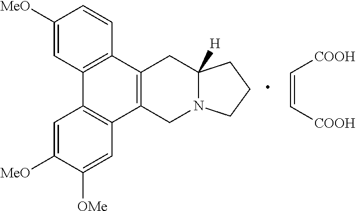

50 mg of (+)-13a-(S)-Deoxytylophorinine (CAT-1) and 16 mg of maleic acid is suspended in 10 mL of ethanol. The mixture is stirred for 10 min in reflux until become clear. The reactant solvent is cold to room temperature and evaporated to 5 mL with yellow solid precipitated. After filtration 45 mg of the maleate salt was obtained. Mp: 126˜128° C. The product is soluble in water.

### Example 3

### Preparation of (+)-13a-(S)-deoxytylophorinine Citrate

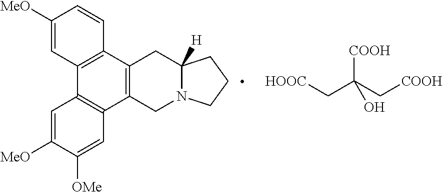

50 mg of (+)-13a-(S)-Deoxytylophorinine (CAT-1) and 29 mg of citric acid is suspended in 10 mL of ethanol. The mixture is stirred for 10 min in reflux until become clear. The reactant solvent is cold to room temperature and evaporated to 5 mL with yellow solid precipitated. After filtration 60 mg of the citrate salt was obtained. Mp: 142˜144° C. The product is soluble in water.

### Example 4

### Preparation of (+)-13a-(S)-deoxytylophorinine Lactate

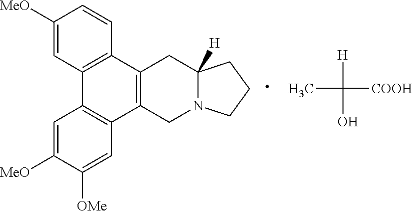

50 mg of (+)-13a-(S)-Deoxytylophorinine (CAT-1) and 14 mg of lactic acid is suspended in 10 mL of ethanol. The mixture is stirred for 10 min in reflux until become clear. The reactant solvent is cold to room temperature and evaporated to 5 mL with yellow solid precipitated. After filtration 45 mg of the lactate salt was obtained. Mp: 115˜117° C. The product is soluble in water.

### Example 5

### Preparation of (+)-13a-(S)-deoxytylophorinine HCl

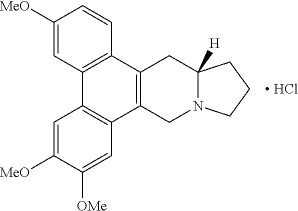

To the 5 mL of dichloromethane solution with 20 mg of (+)-13a-(S)-Deoxytylophorinine (CAT-1) was bubbled with dry HCl gas for 30 min. The reaction solvent was evaporated to get 20 mg of white solid. Mp: 225˜227° C.

### Example 6

### Preparation of (+)-13a-(S)-deoxytylophorinine Sulfate

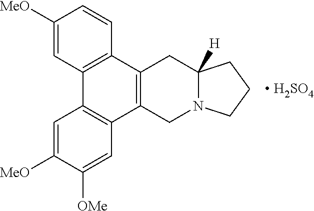

The mixture of 1 g of (+)-13a-(S)-Deoxytylophorinine (CAT-1) in 5 mL of ethanol was dropped with 3 mL of 20% (V/V) sulfuric acid ethanol solution with stiffing. The mixture was heated to reflux until become clear and then cold to room temperature with white solid precipitated. After filtration 1.1 g of the sulfate salt was obtained. Mp: 175˜177° C.

Solubility in water:

| | | +CAT | +CAT | +CAT |
|---|---|---|---|---|
| Sample | +CAT | Citrate | Tartrate | Maleate |
| Solubility) | Insoluble | 33 mg/mL | 39 mg/mL | 44 mg/mL |

| Sample | +CAT Lactate | +CAT Sulfate |
|---|---|---|
| Solubility | 120 mg/mL | 200 mg/mL |

### Pharmacologic Experiments

### Example 1

### Cytotoxic Activity Assay (MTT)

To evaluate the in vitro antitumor activity of (+)-13a-(S)-Deoxytylophorinine salts in this invention, Cytotoxic activity assay (MTT) experiment was persued.

1. Tumor cells at log phase were incubated on 96-well plates at a density of 1˜1.0×10⁴ cells/ml, 100 μl/well. Cells were cultured for 24 h at 37° C. in a cell incubator with 5% CO₂.

2. The treatment cells were exposed to the tested agents at different concentrations and cultured for 5 days in a cell incubator with full humidity incubator.

3. The incubation medium was removed, then 100 μl 0.04% MTT was added to each well and Cells were cultured for 4 hours under the same conditions.

4. The incubation medium was removed, then 150 μl DMSO was added to each well, the absorbance at a reference wavelength of 450 nm and a test wavelength of 570 nm was read. Drug inhibitory rate was calculated.

TABLE 1
MTT result of 13a-(S)-deoxytylophorinine salts:

| | IC₅₀ (M) | | | | | | |
|---|---|---|---|---|---|---|---|
| Samples | A549 | Bel7402 | U251 | BT325 | A2780 | Bgc823 | Hct8 |
| Tartrate | 6.30 × | 6.00 × | 1.30 × | 5.0 × | 8.1 × | 6.3 × | 7.9 × |
| | 10⁻⁰⁸ | 10⁻⁰⁸ | 10⁻⁰⁷ | 10⁻⁰⁷ | 10⁻⁰⁸ | 10⁻⁰⁷ | 10⁻⁰⁸ |
| Maleate | 1.40 × | 3.20 × | 6.30 × | 5.9 × | 5.0 × | 8.9 × | 5.8 × |
| | 10⁻⁰⁷ | 10⁻⁰⁸ | 10⁻⁰⁸ | 10⁻⁰⁷ | 10⁻⁰⁸ | 10⁻⁰⁷ | 10⁻⁰⁸ |
| Lactate | 1.40 × | 4.00 × | 9.50 × | 7.6 × | 1.8 × | 1.3 × | 7.9 × |
| | 10⁻⁰⁷ | 10⁻⁰⁸ | 10⁻⁰⁸ | 10⁻⁰⁷ | 10⁻⁰⁷ | 10⁻⁰⁶ | 10⁻⁰⁸ |
| Citrate | 1.30 × | 3.70 × | 6.90 × | 4.1 × | 4.9 × | 4.0 × | 4.1 × |
| | 10⁻⁰⁷ | 10⁻⁰⁸ | 10⁻⁰⁸ | 10⁻⁰⁸ | 10⁻⁰⁸ | 10⁻⁰⁸ | 10⁻⁰⁸ |
| (+)-13a-(S)- | 4.00 × | 3.80 × | 7.10 × | 5.4 × | 5.4 × | 5.1 × | 5.5 × |
| deoxytylo- | 10⁻⁰⁸ | 10⁻⁰⁸ | 10⁻⁰⁸ | 10⁻⁰⁷ | 10⁻⁰⁸ | 10⁻⁰⁷ | 10⁻⁰⁸ |
| phorinine | | | | | | | |

A549: human lung cancer cell;
Bel7402: human liver cancer cell;
U251: human glioma cell;
BT325: human glioma cell,
A2780: human ovarian cancer cell,
Bgc823: human stomach cancer cell,
Hct8: human colon cancer cell.

The MTT result revealed that the salts of 13a-(S)-deoxytylophorinine displayed notable anti-tumor activity as the same as their prototype.

### Example 2.1

### In Vivo Pharmacodynamic Experiment of 13a-(S)-Deoxytylophorinine and its Salts

To determine the pharmacodynamic characters of 13a-(S)-deoxytylophorinine and its salts, the impact of these compounds to the tumor growth in H22 tumor-bearing mice was surveyed.

### 1. Experimental Animal

Male Kunming mice were obtained from the Laboratory Animal Center, Academy of Military Medical Science. Body weight 18˜22 g. 6 animals per group, totally 10 groups, 60 animals.

### 2. Experimental Samples and Dosage.

(1). Experimental samples

(+)-13a-(S)-deoxytylophorinine, (+)-13a-(S)-deoxytylophorinine Tartrate, (+)-13a-(S)-deoxytylophorinine Maleate, (+)-13a-(S)-deoxytylophorinine Citrate, Cytoxane (CTX) as positive control.

(+)-13a-(S)-deoxytylophorinine, MW: 363.45

(+)-13a-(S)-deoxytylophorinine Tartrate, MW: 513.54

(+)-13a-(S)-deoxytylophorinine Maleate, MW: 479.52

(+)-13a-(S)-deoxytylophorinine Citrate, MW: 573.59

(2) Dosage

(+)-13a-(S)-deoxytylophorinine was administered at high dose (5 mg/kg) and low dose (2.5 mg/kg). Three (+)-13a-(S)-deoxytylophorinine salts were administered at high dose and low dose as equal moles with their prototype (Tartrate: 7.05 mg/kg, 3.53 mg/kg; Malate: 6.60 mg/kg, 3.30 mg/kg; Citrate: 7.90 mg/kg, 3.95 mg/kg).

3. Method

Animals were reared in SPF grade circumstance to be observed for 24 h. The tumor cells liquid which resuscitated in abdominal cavity of KM mice was diluted with aseptic normal saline at 1:3 ratio. The resulting liquid was inoculated subcutaneously at forelimb with 0.2 mL every animal. After the injection over, the animals were divided to 12 groups with 6 animals per group.

Four experimental samples were intragastric administered once daily 24 h after inoculation. The positive control CTX was intraperitoneal injected once daily. The animal body weight was recorded every day.

4. Result

TABLE 2
Impact of (+)-13a-(S)-deoxytylophorinine and its
salts to the tumor growth of H22 tumor-bearing mice.

| | | Animal | | | |
|---|---|---|---|---|---|
| | | No. | | | |
| | Dose | (Initial/ | Body Weight(g) | Tumor Weight | Inhibition |
| Group | (mg/kg × d) | Final) | (Initial/Final) | (g, mean ± SD) | (%) |
| Control | | 6/6 | 21.83 ± 1.47/ | 1.15 ± 0.35 | |
| | | | 25.35 ± 2.76 | | |
| CTX (i.p.) | 100 × 1 | 6/6 | 21.33 ± 1.63/ | 0.59 ± 0.26 | 48.47* |
| | | | 23.77 ± 1.38 | | |
| (+)-13a-(S) | 5.0 × 7 | 6/4 | 23.50 ± 2.51/ | 0.30 ± 0.12 | 74.02** |
| — | | | 15.63 ± 2.16 | | |
| deoxy- | | | | | |
| tylophorinine-1 | | | | | |
| (+)-13a-(S) | 2.5 × 7 | 6/6 | 23.00 ± 1.55/ | 0.66 ± 0.21 | 42.79* |
| — | | | 23.31 ± 2.14 | | |
| deoxy- | | | | | |
| tylophorinine-2 | | | | | |
| Tartrate-1 | 7.05 × 7 | 6/6 | 22.00 ± 1.55/ | 0.23 ± 0.15 | 80.35** |
| | | | 13.55 ± 1.87 | | |
| Tartrate-2 | 3.53 × 7 | 6/6 | 21.50 ± 0.84/ | 0.78 ± 0.48 | 31.88 |
| | | | 19.76 ± 2.34 | | |
| Maleate-1 | 6.60 × 7 | 6/4 | 21.17 ± 0.98/ | 0.30 ± 0.24 | 73.58** |
| | | | 13.62 ± 2.08 | | |
| Maleate-2 | 3.30 × 7 | 6/6 | 22.50 ± 1.05/ | 0.62 ± 0.31 | 45.56 |
| | | | 23.79 ± 0.52 | | |
| Citrate-1 | 7.90 × 7 | 6/5 | 22.00 ± 1.55/ | 0.22 ± 0.13 | 80.44** |
| | | | 14.27 ± 2.61 | | |
| Citrate-2 | 3.95 × 7 | 6/6 | 21.83 ± 0.75/ | 0.61 ± 0.35 | 46.87* |
| | | | 21.01 ± 2.04 | | |

*p < 0.05,
**p < 0.01 compared with control group.

5. Conclusion

The inhibition of organic acid salts of (+)-13a-(S)-deoxytylophorinine to tumor growth of animal was equal or higher with their prototype at high dose, equal or lower at low dose, but all were higher than positive control (CTX), which means the anti-tumor activity was retained after salification.

### Example 2.2

### In Vivo Pharmacodynamic Experiment of 13a-(S)-deoxytylophorinine

### Sulfate

### 1. Purpose

To observe the impact of 13a-(S)-deoxytylophorinine Sulfate: on the tumor growth of H22 tumor-bearing mice.

### 2. Experimental Animal

Male Kunming mice were obtained from the Laboratory Animal Center, Academy of Military Medical Science. Body weight 18˜22 g.

### 3. Experimental Samples and Dosage.

(1). Experimental Samples

(+)-13a-(S)-deoxytylophorinine Sulfate, Cytoxane (CTX) as positive control.

(2) Dosage and Administration Way

All the samples were administered orally. o.d. 0.2 ml/10 g.

(+)-13a-(S)-deoxytylophorinine Sulfate was administered at five dose groups: 3 mg/kg, 4 mg/kg, 5 mg/kg, 6 mg/kg and 8 mg/kg.

### 4. Method

Animals were reared in SPF grade circumstance to be observed for 24 h. The tumor cells liquid which resuscitated in abdominal cavity of KM species mice was diluted with aseptic normal saline at 1:3 ratio. The resulting liquid was inoculated subcutaneously at forelimb with 0.2 mL every animal. After the injection over, the animals were divided to 13 groups. Experimental samples were administered 24 h after inoculation.

### 5. Result

TABLE 1
The impact of (+)-13a-(S)-deoxytylophorinine sulfate
to the tumor growth of H22 tumor-bearing mice.

| | | Animal | | | |
|---|---|---|---|---|---|
| | | No. | | Tumor | |
| | Dose | (Initial/ | Body Weight(g) | Weight | Inhibition |
| Group | (mg/kg × d) | Final) | (Initial/Final) | (g, mean ± SD) | (%) |
| Control | | 6/6 | 19.67 ± 1.21/ | 1.26 ± 0.22 | |
| | | | 26.17 ± 5.38 | | |
| CTX (i.p.) | 100 mg/kg × 1 | 6/6 | 20.67 ± 2.07/ | 0.07 ± 0.04 | 94.76** |
| | | | 25.00 ± 2.37 | | |
| Sulfate | 3 mg/kg × 7 | 6/6 | 20.67 ± 0.52/ | 0.98 ± 0.21 | 22.49* |
| (o.p.) | | | 26.83 ± 2.56 | | |
| Sulfate | 4 mg/kg × 7 | 6/6 | 20.00 ± 1.26/ | 0.60 ± 0.32 | 52.51* |
| (o.p.) | | | 22.83 ± 2.40 | | |
| Sulfate | 5 mg/kg × 7 | 6/6 | 19.83 ± 1.72/ | 0.73 ± 0.39 | 41.75* |
| (o.p.) | | | 22.17 ± 5.31 | | |
| Sulfate | 6 mg/kg × 6 | 6/3 | 19.83 ± 1.72/ | 0.40 ± 0.24 | 68.52** |
| (o.p.) | | | 15.33 ± 3.21 | | |
| Sulfate | 8 mg/kg × 6 | 6/0 | 20.67 ± 1.03/ | 0.15 ± 0.04 | 88.49** |
| (o.p.) | | | — | | |

Note:
After (+)-13a-(S)-deoxytylophorinine Sulfate (6 mg/kg) was administered orally, one of mice died on sixth day, two of mice died on seventh day. Stop administion.
After (+)-13a-(S)-deoxytylophorinine Sulfate (8 mg/kg) was administered orally, one of mice died on sixth day, two of mice died on seventh day. Stop administion.
The tumor weight data and the inhibition rate of these groups is obtioned after (+)-13a-(S)-deoxytylophorinine Sulfate was administered orally 7 days and then the mice was dissected.
*p < 0.05,
**p < 0.01 compared with control group.

### Example 3

### The Study about Plasma Pharmacokinetics and Distribution in Brain Tissue of 13a-(S)-deoxytylophorinine and its Salts

To determine the pharmacokinetic characters of 13a-(S)-deoxytylophorinine and its salts, the studies about plasma pharmacokinetics and the character of distribution in brain were pursued.

### 1. Experimental Animal

Male ICR mice. Body weight 18˜22 g.

### 2. Samples

(+)-13a-(S)-deoxytylophorinine Tartrate, (+)-13a-(S)-deoxytylophorinine Maleate, (+)-13a-(S)-deoxytylophorinine Citrate. All were dissolved in distilled water as 0.6 mg/ml solution before experiment progressed.

### 3. Method

72 mice were divided to 3 groups with 24 animals per group, fasting for 16 h and drinking water freely. After the salts were administered orally, the blood of mice were collected at 5 min, 15 min, 30 min, 1 h, 2 h, 3 h, 4 h, 6 h time points. Plasma was isolated by centrifugalization. After the salts were administered orally, the brain tissues of mice were collected at 5 min, 15 min, 2 h time points. 25% tissue homogenate was prepared with normal saline addition. 200 μL of homogenate and plasma were added isovolumic MeCN to precipitate protein. 5 μL of clear supernatant was analyzed by LC/MS/MS.

### 4. Result

### (1). Standard Curve

(+)-13a-(S)-deoxytylophorinine was dissolved in DMSO and then diluted with MeOH with concentration 10, 50, 100, 500, 1000 and 2500 ng/ml respectively. 10 μl of standard solutions of (+)-13a-(S)-deoxytylophorinine with different concentration above were added to 200 μl of blank plasma sequently to adjust the concentration to 0.5, 2.5, 5, 25, 50, 125 ng/ml respectively. MeCN was added to the solution to precipitate protein, and 5 μl of result clear supernatant was analyzed with LC/MS/MS. Linear regression was ran with the peak area of (+)-13a-(S)-deoxytylophorinine as ordinate and the concentrations as abscissa. The result was represented in Table 3 and FIG. 4. In the scope of concentration with 0.5-125 ng/ml, the linear relation of the concentrations of (+)-13a-(S)-deoxytylophorinine in plasma and peak area was fine with correlation coefficient 0.999.

TABLE 3
Standard curve of (+)-13a-(S)-deoxytylophorinine in mice plasma
Samples in plasma

| Concentration (ng/ml) | Peak area |
|---|---|
| 0.5 | 2707 |
| 2.5 | 16732 |
| 5 | 34240 |
| 25 | 169854 |
| 50 | 310165 |
| 125 | 785030 |

(2). Plasma Pharmacokinetics Study of Three (+)-13a-(S)-Deoxytylophorinine Salts After Administration Orally by Mice.

The concentration-time data of (+)-13a-(S)-deoxytylophorinine tartrate, maleate and citrate after administration orally were represented in Table 4˜6 and FIG. 5. These salts were absorbed quickly, the prototype compound was detected in blood in 5 min after administration orally. The peak concentration of tartrate was 34.2±3.1 ng/ml after 15 min. The peak concentration of maleate was 35.4±3.5 ng/ml after 5 min. The peak concentration of citrate was 20.1±12.2 ng/ml after 15 min. The elimination of all the salts were quickly with blood drug levels approached lowest detectable limit after 6 h. The pharmacokinetic parameters were represented in Table 7 which fitted with non-compartment model by using DAS program according to the concentration-time curve of (+)-13a-(S)-deoxytylophorinine salts. MRT (0-∞) of these salts is 1.63˜2.29 h, which is longer than their prototype (0.8 h). The AUC(0-∞) of tartrate, maleate and citrate is 37.85, 49.91 and 33.08 ug/L*h respectively, compared with their prototype 33.67 ug/L*h. The AUC(0-∞) of maleate is higher than other salts and prototype compound notably.

TABLE 4
The drug concentration of (+)-13a-(S)-deoxytylophorinine
tartrate administered orally (6 mg/kg, 11.7 uM)

| | |
|---|---|
| | Blood concentration of (+)-13a-(S)- |
| Time | deoxytylophorinine (ng/ml) |

| (min) | 1 | 2 | 3 | Mean ± SD |
|---|---|---|---|---|
| 0.08 | 18.4 | 11.7 | 42.0 | 24.0 ± 15.9 |
| 0.25 | 30.6 | 36.1 | 35.7 | 34.2 ± 3.1 |
| 0.5 | 16.5 | 9.0 | 7.8 | 11.1 ± 4.7 |
| 1 | 6.2 | 10.0 | — | 8.1 ± 2.7 |
| 2 | 2.5 | 12.4 | 8.7 | 7.9 ± 5.0 |
| 3 | 3.0 | 5.0 | 5.3 | 4.5 ± 1.2 |
| 4 | 3.3 | 4.2 | 1.1 | 2.8 ± 1.6 |
| 6 | 0.0 | 0.2 | 0.8 | 0.4 ± 0.4 |

TABLE 5
The drug concentration of (+)-13a-(S)-deoxytylophorinine
maleate administered orally (6 mg/kg, 12.5 uM)

| | |
|---|---|
| | Blood concentration of (+)-13a-(S)- |
| Time | deoxytylophorinine (ng/ml) |

| (min) | 1 | 2 | 3 | Mean ± SD |
|---|---|---|---|---|
| 0.08 | — | 33.0 | 37.9 | 35.4 ± 3.5 |
| 0.25 | 24.2 | 23.5 | 34.6 | 27.4 ± 6.2 |
| 0.5 | 15.7 | 13.0 | 7.9 | 12.2 ± 4.0 |
| 1 | 12.5 | 6.2 | 8.7 | 9.2 ± 3.2 |
| 2 | 2.0 | 15.9 | 15.7 | 11.2 ± 8.0 |
| 3 | 3.6 | 16.1 | 6.3 | 8.6 ± 6.6 |
| 4 | 2.6 | 2.0 | 3.8 | 2.8 ± 0.9 |
| 6 | 2.3 | 1.0 | 0.4 | 1.2 ± 1.0 |

TABLE 6
The drug concentration of (+)-13a-(S)-deoxytylophorinine citrate
administered orally (6 mg/kg, 10.5 uM)

| | |
|---|---|
| | Blood concentration of (+)-13a-(S)- |
| Time | deoxytylophorinine (ng/ml) |

| (min) | 1 | 2 | 3 | Mean ± SD |
|---|---|---|---|---|
| 0.08 | 30.5 | 18.0 | 4.6 | 17.7 ± 13.0 |
| 0.25 | 25.5 | 28.7 | 6.1 | 20.1 ± 12.2 |
| 0.5 | 20.1 | 8.4 | 9.3 | 12.6 ± 6.5 |
| 1 | 11.2 | 5.4 | 8.5 | 8.4 ± 2.9 |
| 2 | 5.6 | 7.6 | 9.6 | 7.6 ± 2.0 |
| 3 | 2.8 | 2.3 | 4.7 | 3.3 ± 1.3 |
| 4 | 1.1 | 0.9 | 2.6 | 1.5 ± 0.9 |
| 6 | 1.4 | 4.8 | 0.1 | 2.1 ± 2.4 |

TABLE 7
Pharmacokinetic parameters of (+)-13a-(S)-deoxytylophorinine
salts administered orally (6 mg/kg)

| | | Parameter value | | | |
|---|---|---|---|---|---|
| Parameter | Unit | Tartrate | Maleate | Citrate | Prototype |
| AUC(0-t) | ug/L*h | 37.39 | 46.87 | 32.70 | 33.34 |
| AUC(0-∞) | ug/L*h | 37.85 | 49.91 | 33.08 | 33.67 |
| MRT(0-t) | h | 1.57 | 1.78 | 1.73 | 0.77 |
| MRT(0-∞) | h | 1.63 | 2.18 | 2.29 | 0.80 |
| t½z | h | 0.88 | 1.72 | 0.86 | 0.44 |
| Tmax | h | 0.25 | 0.08 | 0.25 | 0.25 |
| Cmax | ug/L | 34.17 | 35.45 | 20.10 | 41.53 |

(3). Drug Distribution Study of Three (+)-13a-(S)-Deoxytylophorinine Salts in Brain Tissue after Administration Orally by Mice.

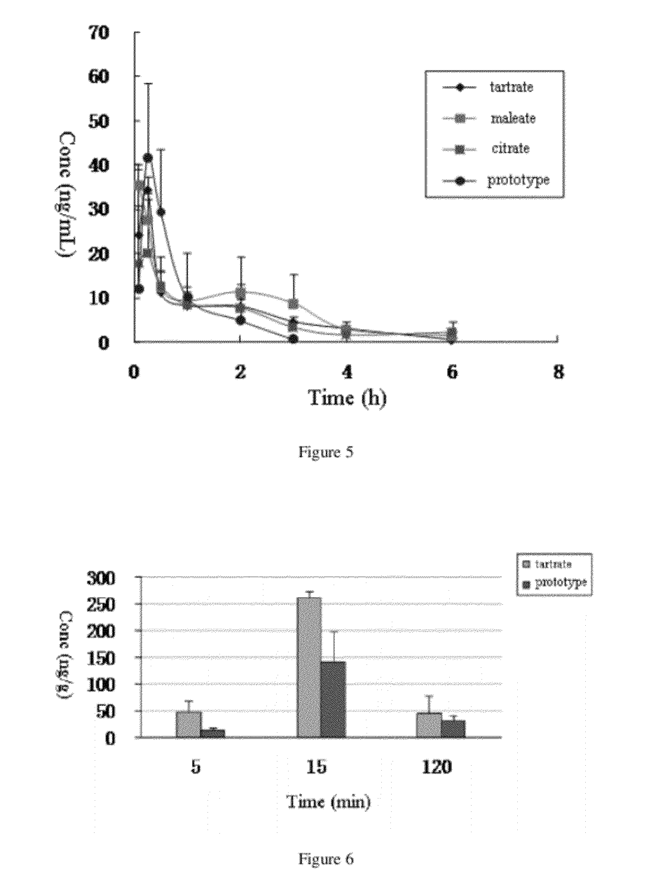

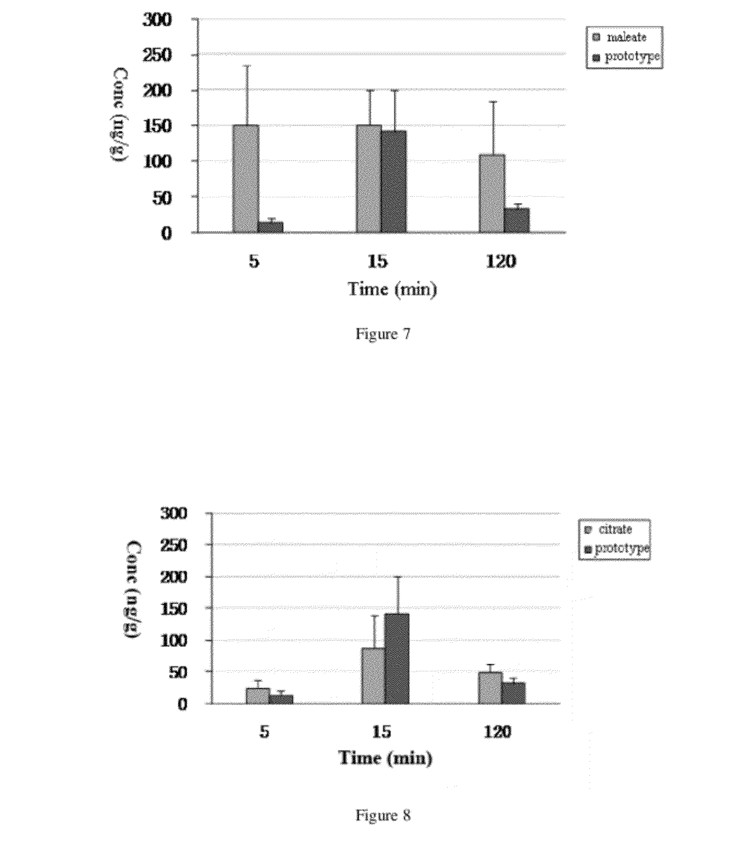

The drug concentration-time data in brain tissue after administered orally of (+)-13a-(S)-deoxytylophorinine tartrate, maleate and citrate were represented in Table 8˜10 and FIG. 6˜8. As the results revealed, the drug could distributed into brain tissue easily after the salts taken orally. (+)-13a-(S)-deoxytylophorinine could be detected in 5 min. The peak concentration was reached after 15 min, and the compound still can be detected after 2 h after administration. The drug contents of tartrate and maleate in brain at 3 time points were all higher than their prototype, and the content of citrate was lower than its prototype.

TABLE 8
The drug contents in brain tissue of (+)-13a-(S)-deoxytylophorinine
tartrate administered orally (6 mg/kg)

| | |
|---|---|
| | Brain contents of (+)-13a-(S)- |
| Time | deoxytylophorinine (ng/ml) |

| (min) | 1 | 2 | 3 | Mean ± SD |
|---|---|---|---|---|
| 5 | 32.4 | 38.8 | 72.7 | 48.0 ± 21.6 |
| 15 | 249.3 | 271.0 | 268.5 | 262.9 ± 11.9 |
| 120 | 24.7 | 28.7 | 83.7 | 45.7 ± 33.0 |

TABLE 9
The drug contents in brain tissue of (+)-13a-(S)-
deoxytylophorinine maleate administered orally (6 mg/kg)

| | |
|---|---|
| | Brain contents of (+)-13a-(S)- |
| Time | deoxytylophorinine (ng/ml) |

| (min) | 1 | 2 | 3 | Mean ± SD |
|---|---|---|---|---|
| 5 | 245.2 | 82.5 | 122.7 | 150.2 ± 84.8 |
| 15 | 159.3 | 96.6 | 194.9 | 150.3 ± 49.8 |
| 120 | 27.2 | 173.7 | 125.4 | 108.8 ± 74.7 |

TABLE 10
The drug contents in brain tissue of (+)-13a-(S)-
deoxytylophorinine citrate administered orally (6 mg/kg)

| | |
|---|---|
| | Brain contents of (+)-13a-(S)- |
| Time | deoxytylophorinine (ng/ml) |

| (min) | 1 | 2 | 3 | Mean ± SD |
|---|---|---|---|---|
| 5 | 22.0 | 37.1 | 11.8 | 23.6 ± 12.7 |
| 15 | 84.8 | 138.7 | 38.5 | 87.3 ± 50.1 |
| 120 | 37.0 | 44.8 | 64.4 | 48.7 ± 14.1 |

### 5. Conclusion

The absorption were quickly after the salts of (+)-13a-(S)-deoxytylophorinine tartrate, maleate and citrate administration orally. The peak concentration was 18.0˜35.4 ng/ml in 5˜30 min MRT (0-∞) of these salts is 1.63˜2.29 h, which is longer than their prototype (0.8 h). The AUC(0-∞) of tartrate, maleate and citrate is 37.85, 49.91 and 33.08 ug/L*h respectively, compared with their prototype 33.67 ug/L*h. The AUC(0-∞) of maleate is higher than other salts and prototype compound notably.

The drug could be distributed into brain tissue easily after the salts oral administration. The peak concentration was detected after 15 min, and the compound still can be detected after 2 h after administration. The drug contents of tartrate and maleate in brain at 3 time points were all higher than their prototype, and the content of citrate was lower than its prototype.

1. The salts of (+)-13a-(S)-deoxytylophorinine with different acid represented by the general formula (I): 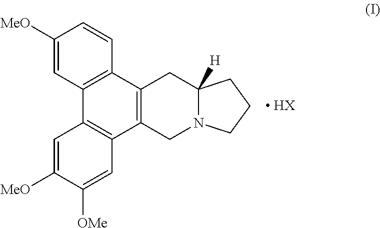
Wherein:
FIX is organic acid or inorganic acid.

2. The salts of (+)-13a-(S)-deoxytylophorinine with different acid according to claim 1, characterized in that, said salts are selected from hydrochloric acid salt, phosphoric acid salt, sulfuric acid salt, hydrobromic acid salt, tartaric acid salt, citric acid salt, maleic acid salt, lactic acid salt, salicylic acid salt, malic acid salt, benzoic acid salt, hexanedioic acid salt, fumaric acid salt, succinic acid salt, sulfonic acid salt, alginic acid salt, amino acid salt, acetyl benzoic acid salt, folic acid salt, N-cyclohexylamino sulfonic acid salt, polygalacturonic acid salt, methane sulfonic acid salt or tosic acid salt.

3. A pharmaceutical composition characterized in comprising a pharmaceutically effective dosage of a compound according to any one of claims 1-2, and a pharmaceutically acceptable carrier.

4. A pharmaceutical composition according to claim 3, characterized in that, said the pharmaceutical composition is selected from tablets, capsules, pills, injections, sustained-release preparation, controlled-release or targeted preparations and various fine particle delivery system.

5. Use of a compound according to any one of claims 1-2 for the preparation of drugs for the prevention and/or treatment of cancer disease.

6. Use according to claim 5, characterized in that, said cancer is selected from human colon cancer, human stomach cancer, human ovarian cancer, uterine cervix cancer, liver cancer, lung cancer, pancreatic cancer, lymphadenoma and glioma.

7. Use of a compound according to any one of claims 1-2 for the preparation of drugs for the prevention and/or treatment of inflammation disease.
